(19) United States
(12) Patent Application Publication
(10) Pub. No.: US 2025/0377330 A1
(43) Pub. Date: Dec. 11, 2025

(54) ELECTROCHEMICAL BIOSENSORS AND METHOD OF MANUFACTURING ELECTROCHEMICAL BIOSENSORS

(71) Applicant: GLC MEDICAL (GLCM) INC., Edmonton (CA)

(72) Inventors: Ushula TEFASHE, Edmonton (CA); Mikolai VAJDA, Edmonton (CA); Brad KRUECKL, Edmonton (CA); Josh GÉNÉREUX, Edmonton (CA)

(21) Appl. No.: 18/877,952

(22) PCT Filed: Jun. 20, 2023
(86) PCT No.: PCT/CA2023/050858

§ 371 Date: Dec. 20, 2024

Related U.S. Application Data
(60) Provisional application No. 63/353,828, filed on Jun. 20, 2022.

(51) Int. Cl. G01N 27/327; G01N 27/30; G01N 33/543

(52) U.S. Cl. CPC G01N 27/3276; G01N 27/308; G01N 33/5438

(57) ABSTRACT

A sensor for detecting an analyte of interest in a fluid sample has a device architecture that includes a working electrode, a reference electrode and a counter electrode. The working electrode has a functionalized carbon surface to target the analyte of interest wherein, in response to a fluid sample applied to the working electrode that includes the analyte of interest, the device architecture generates an electrical characteristic indicative of the analyte of interest. The substrate comprises a material that is resistant to multiple heating cycles during which the substrate is heated to a temperature of between 100-150° C. for at least 10 minutes.

### TECHNICAL FIELD

This relates to electrochemical biosensors, and in particular, electrochemical biosensors with printed metal and carbon electrodes.

### BACKGROUND

With the recent global pandemic, efforts towards the development of rapid tests for viruses and infection by-products have led to a general push to improve sensing technology for diagnostic applications. In these devices, binding events at the surface of a material with targeted functionality leads to a change in a measurable physical property such as colour, fluorescence, or electrochemical signal.

### SUMMARY

According to an aspect, there is provided a sensor for detecting an analyte of interest in a fluid sample, comprising a substrate that supports a device architecture, the device architecture comprising a working electrode, a reference electrode and a counter electrode, the working electrode comprising a functionalized carbon surface to target the analyte of interest wherein, in response to a fluid sample applied to the working electrode that includes the analyte of interest, the device architecture generates an electrical characteristic indicative of the analyte of interest, wherein the substrate comprises a material that is resistant to multiple heating cycles during which the substrate is heated to a temperature of between 100-150° C. for at least 10 minutes

According to other aspects, the sensor may comprise one or more of the following features, alone or in combination: the sensor may further comprise a sensing region that is adapted to receive the fluid sample in electrical communication with the reference and counter electrode; the functionalized carbon surface may comprise amorphous carbon, carbon black, graphite, exfoliated graphite, graphene nanoplatelets, binders, stabilizers, or combinations thereof; each functionalized carbon surface may be functionalized with linkers, each linker having a first terminus that is bound to the working electrode and a second terminus that is bound to a biorecognition element, each biorecognition element being selected to bind a predetermined analyte; a surface of the substrate may be covalently functionalized with a benzoic acid-based linker through diazonium reduction reaction of the linker via the substrate under an applied voltage; the linkers may comprise an ester group and are electrografted to the functionalized carbon substrate; the linkers may be bonded to an antibody or biorecognition element; the sensor may further comprise a blocking agent to block a portion of the functionalized carbon surface sensor areas not covered with the antibody or biorecognition element; and the detector may be adapted to detect changes using electrochemical impedance spectroscopy measurement.

According to an aspect, there is provided a method of detecting an analyte of interest, comprising the steps of: applying a sample solution to the sensing region and allowing the sample to interact for a predetermined period of time; applying a test solution covering all the three electrodes resulting in electrical communication of the sensors; and acquiring EIS measurements and assessing if a measurable EIS shape and size is indicative of the presence or absence of the analyte of interest.

According to an aspect, there is provided a method of manufacturing a biosensor, comprising: printing electrodes on a substrate using metallic ink; curing the electrodes at a temperature of between 100-150° C. for at least 10 minutes; printing a carbon surface using carbon-containing ink, the carbon surface being in electrical communication with the electrodes; curing the carbon surface at a temperature of between 100-150° C. for at least 10 minutes; and functionalizing the carbon surface to target an analyte of interest, such that the electrodes generate an electrical characteristic in response to the analyte of interest being applied to the functionalized carbon surface.

According to other aspects, the method may comprise one or more of the following features, alone or in combination: the method may further comprise the step of applying a dielectric layer to the electrodes and curing the dielectric layer; the functionalized carbon surface may comprise a sensing region that is adapted to receive the fluid sample; the carbon ink may comprise amorphous carbon, carbon black, graphite, exfoliated graphite, graphene nanoplatelets, binders, stabilizers, or combinations thereof; the functionalized carbon surface may be functionalized with linkers, each linker having a first terminus that is bound to a working electrode and a second terminus that is bound to a biorecognition element, each biorecognition element being selected to bind a predetermined analyte; a surface of the substrate may be covalently functionalized with a benzoic acid-based linker through diazonium reduction reaction of the linker via the substrate under applied voltage; method may further comprise chemical modification of the linker to introduce an ester group following electrografting to the surface; the method may further comprise bonding the chemically modified linker to an antibody or biorecognition element, and incubating the biosensor for at least 24 hours at a temperature of 3-8° C.; the method may further comprise applying a blocking agent to block sensor areas not covered with antibody to reduce the non-specific binding of analyte; applying the blocking agent may comprise a 1 hr incubation time at room temperature with polyethylene glycol 8000 Da as the blocking agent; the detector may be adapted to detect changes using electrochemical impedance spectroscopy measurement; and the biosensor may be packaged in a sensor vessel, vacuum sealed and stored at 3-8° C. for at least 2 months.

According to an aspect there is provided a sensor, methods of manufacture, and methods of detecting an analyte of interest as defined in the claims.

In other aspects, the features described above may be combined together in any reasonable combination as will be recognized by those skilled in the art.

### BRIEF DESCRIPTION OF THE DRAWINGS

These and other features will become more apparent from the following description in which reference is made to the appended drawings, the drawings are for the purposes of illustration only and are not intended to be in any way limiting, wherein:

FIG. 1 is a flowchart of the fabrication process for printing electrodes on a substrate to make a biosensor.

FIG. 2 is a flowchart showing the fabrication process and working principle of a sensor that uses electrochemical impedance spectroscopy detection.

FIG. 3 is a circuit diagram that models a biosensor.

### DETAILED DESCRIPTION OF PREFERRED EMBODIMENTS

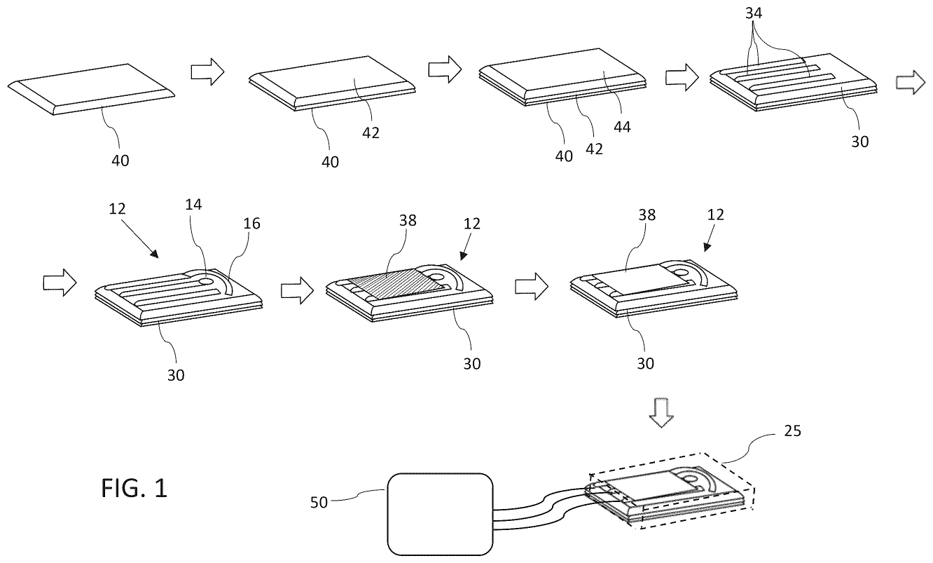

A sensor, generally identified by reference number 10, will now be described with reference to FIG. 1 through 3. Sensor 10 is a biosensor with highly reproducible electrochemical characteristics through electrochemical cleaning and functionalization of screen-printed carbon electrodes.

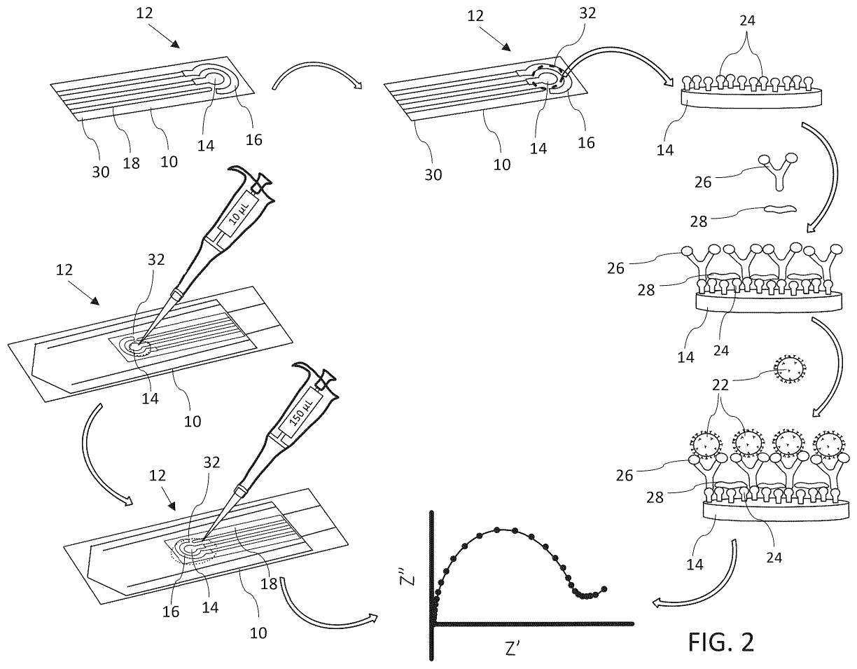

Referring to FIG. 1, sensor 10 supports a device architecture 12. Device architecture includes a working electrode 14, a counter electrode 16, and a reference electrode 18. Referring to FIG. 2, working electrode 14 has a functionalized carbon surface that has been functionalized, such as with linker molecules 24, to target an analyte of interest 22. The electrodes 14, 16, and 18 may be screen printed carbon. The screen-printed carbon electrodes may be produced using inks which contain amorphous carbon, carbon black, graphite, exfoliated graphite, graphene nanoplatelets, or combinations thereof and may include binders, stabilizers, or other additives to improve the properties of the ink. The carbon ink may be primarily sp² carbon. While the use of a screen-printed carbon electrode is discussed in detail below, the carbon surface may also be generated by coating an electrode made from a different material with carbon ink, provided that the carbon surface is compatible with the functionalization chemistry described below.

The performance of the sensor depends in part on the density of a surface bound linker 24 that tethers analyte of interest 22, which may be a biomolecule, to the surface of working electrode 14, and the amount of surface left exposed. The nature of the chosen linker 24 may depend on several factors including what the desired chemical properties of the linker are to bind it to a biological recognition element 26, and the ability to bind to the surface of working electrode 14 through electrografting under applied potential. Since the tethering of analyte of interest 22 to the surface is reliant only on the presence of a primary amine, the family of molecules to be used as biological recognition element 26 may include oligopeptides and polypeptides, including proteins, enzymes, and in some examples, antigens or antibodies, though the technology need not be limited to biomolecules. The surface of working electrode 14 may be functionalized with a single biological recognition element 26 or may be functionalized with several molecules which may be selected to target different analytes, or different regions of the same analyte.

To perform the diagnostic test, a detector may be used to transduce the binding event into a usable signal, which in this case is intended for use in electrochemical measurements. The selection of the appropriate electrochemical technique may be determined empirically but may be reliant on how binding to the surface changes electrical characteristics of the surface/interface on completion of the binding event. In one example, the sensor may be used in electrochemical impedance spectroscopy (EIS); a technique that is suitable for use with screen printed electrochemical sensors. The reason for selecting this technique is that it depends on the nature and chemical properties of the interface between the electrode surface and the electrolyte solution which means that any change at the surface will affect parameters such as the capacitance of the electrical double layer formed at the surface of the electrode, or a change in the charge transfer resistance relating to a redox couple selected to amplify the electrochemical impacts of binding. The species chosen may not otherwise interfere with the electrode surface, and may be relatively inert and have predictable redox chemistry. Suitable candidates for the redox couple may include ferric/ferrocyanide solution which is commonly used in EIS for biosensing applications.

In electrochemical impedance detection, beneficial results are generally achieved by covering the surface of an electrochemical biosensor as thoroughly as possible which serves two overall purposes: to maximize the number of available active sites for binding to the analyte to occur, and to minimize the amount of surface left uncovered to allow for non-specific binding to either the analyte, or other elements present in the biological samples under test. To achieve this goal, the proposed approach involves 3-fold strategy involving acidic and basic cleaning of the surface under applied potential, and then electrografting of the surface with a covalently bonded linker molecule, whose purpose is to provide a chemical handle that can then be bonded to any molecule, nanoparticle or microparticle that would provide an appropriately structured site for selective binding to the analyte of interest (the biological recognition element such as antibodies, aptamers, enzymes, or polypeptide molecules or fragments).

The method of manufacturing the proposed sensors is fundamentally flexible and while the discussion below is in the context of sensors used to detect SARS-CoV-2 in saliva, the surface may be tailored to detect other viruses, such as known viruses (e.g., SARS-CoV-1, MERS, HIV, Zika, etc.), or future viruses that have not yet evolved or presented.

Generally, a screen-printed electrochemical sensor 10 comprises a device architecture 12 that includes several electrodes printed on a substrate 30. as the electrodes include working electrode 14, counter electrode 16, and reference electrode 18 that work together with a potentiostat to apply electrical signal through an electrochemical cell containing electrolyte solution. The use of three or more electrodes may reduce the voltage drop in the electrochemical cell and improve the accuracy of electrochemical detection signal. As shown in FIG. 2, device architecture 12 may include a sensing region 32 that includes working electrode 14, counter electrode 16, and reference electrode 18, and is designed to receive a test sample that is to be tested for the presence of an analyte of interest and a conductive fluid that places electrode 14, 16, 18 in electrical communication to test for the presence of any analyte that may be bound to the functionalized surfaces. A test signal may be applied to sensing region 32 using a signal generator (not shown) that applies an AC voltage that may have a variable frequency. The voltage difference is applied between working electrode 14 and counter electrode 16, while reference electrode 18 provide a reference for the voltage difference between electrodes 14 and 16.

In one example, Melinex® ST505 (source: DuPont) was used as substrate 30 for sensor 10. Substrate 30 consists of 500 μm thick film layer of PET-adhesive-PET (polyethylene terephthalate). Silver ink was used to print electrical tracks 34 and reference electrode 18 (thickness 7±1 μm), while working and counter electrodes 14 and 16 (thickness 20±2 μm) were printed with carbon ink. A UV curable dielectric and varnish material 38 (such as may be obtained, for example from Fujifilm™) was printed to the exposed silver electrical tracks 34 preventing it from contact with electrolyte solution. Each sheet of printed circuit consists of 120 sensors.

An example of a production process is shown in FIG. 1, starting with a bottom PET layer 40, to which an adhesive layer 42 and a top PET layer 44 were added to form substrate 30. Silver ink was then printed on the substrate and allowed to cure at about 125° C. for about 10 minutes to form electrical tracks 34. Carbon ink was then printed on substrate and allowed to cure at about 125° C. for about 10 minutes to form working and counter electrodes 14 and 16. Two coats of dielectric ink were applied with each coat UV cured. A transparent varnish coat was then applied, and UV cured.

In general, substrate 30 is selected for its stability at high temperatures. Preferably substrate 30 should be able to withstand temperatures of between 100-150° C. for over 10 minutes of repeated exposure. In some cases, substrate 30 may be subjected to four or more heating cycles throughout the manufacturing process. A suitable substrate 30 may include one or more polymeric layers, such as PET as discussed above, that are adhered together and that are stable to the expected temperatures. In some examples, there may be 2, 3, or more layers, which may improve the structural stability of substrate 30. This allows the inks to be cured at higher temperatures and for longer periods of time. In addition, substrate 30 is selected to have a sufficiently high dielectric constant to prevent shorting between electrodes or coated with a suitable dielectric material prior to printing the electrodes.

The metal ink, such as silver ink, is selected due to its good conductivity, good resistance to organic and inorganic solvent and excellent adhesion to the surface and is cured at 125° C. for about 10 minutes, which results in stable and highly conductive sliver ink tracks 34. The carbon ink, which may be any of those discussed above, is also cured such that solvent gets removed. A low porosity dielectric coating and transparent varnish print was UV cured to achieve excellent adhesion of the coating to the surface.

Once substrate 30 is prepared, the carbon working electrode 14 surfaces may be functionalized using a linking molecule 24 through diazotization reaction, which is then chemically converted to possess the right terminal group such that it can be bonded to biological recognition elements 26 such as antibodies, aptamers, enzymes, or polypeptide molecules or fragments. For example, the printed electrodes may be covalently functionalized with a benzoic acid-based linker through a diazonium reduction reaction of the linker with the substrate or electrode under an applied voltage. In some examples, the linker may include an ester group. The linkers may be electrografted to the functionalized carbon substrate. In one example, aryldiazonium salts prepared from aminobenzoic acid are chosen that generate good surface coverage and provide the carboxylic acid group which is readily converted to N-Hydroxysuccinimide ester for subsequent reaction to primary amines present on the biomolecules which form biological recognition elements 26 for the sensors described. These biological recognition elements 26 may then bind to, or otherwise react with, biological analytes of interest 22 such as antigens, viruses, biomarkers, hormones, or bacterial debris. In using an electrochemical grafting technique for functionalizing the linker group 24 on the surface, more continuous surface coverage is possible than common non-covalent approaches such as using pi-pi stacking to functionalize the surface, leading to better surface coverage, and stronger bonding to biological recognition elements 26 generating a more robust sensing surface.

An antibody or biorecognition element 26 may be incubated on the functionalized working carbon electrode 14 and stored in an airtight container at 4° C. fridge for 24 hours. The 24 hours incubation of the antibody ensures formation of a strong chemical bond between biological recognition elements 26 and linker 24 and generate a homogenously covered sensor surface, resulting in a reproducible sensor production. The biosensor may then be packaged in a sensor vessel 25. In some examples, the biosensor may be vacuum sealed and suitable for storage for 2 months or more when maintained at a temperature of around 3-8° C.

To reduce non-specific binding of analyte, the sensors functionalized with antibody may be incubated with a blocking agent 28 in order to passivate the exposed surfaces of the sensor. The blocking agent may be polyethylene glycol 8000 Da.

The combination of these processes may be used to achieve a reproducible and commercially viable production of electrochemical impedance spectroscopy-based biosensor. It has been found that the tests performed on the analyte in biological matrix in the presence of a redox couple improves signal strength.

Referring to FIG. 2, an example of a process by which a sensor is manufactured and used is shown, starting with a bare screen-printed carbon electrode (SPCE). Working electrode 14 is smudged and then reacted with a linking molecule 24, biological recognition elements 26, and blocking agent 28 conjugation suitable for conjugation with a target biomolecule, such as a virus or bacteria. In one example, a 500 μm thick PET-adhesive-PET substrate 30 was used to print a three-electrode electrochemical biosensor device. The PET-adhesive-PET substrate 30 consisted of two layers of 185 μm thick ST505 PET film and a 130 μm thick adhesive layer. The PET-adhesive-PET substrates displayed higher resistance to repeated cycle of high temperature (125° C.), exposure to print metals and carbon inks, and were able to remain stable for organic and inorganic solvent treatment and were found to have a high and low pH resistance. The biosensor manufactured using this approach was found to be reproducible, with adequate levels of sensitivity, and stability. A dielectric layer 38 of the black dielectric ink and a transparent varnish material were applied and cured with repeated cycle of UV exposure. These layers were found to be biocompatible and did not leak, release toxic molecules, or degrade during electrochemical impedance spectroscopy detection. The application of double dielectric layer and a transparent varnish layer was found to be stable towards water and alcohol treatment.

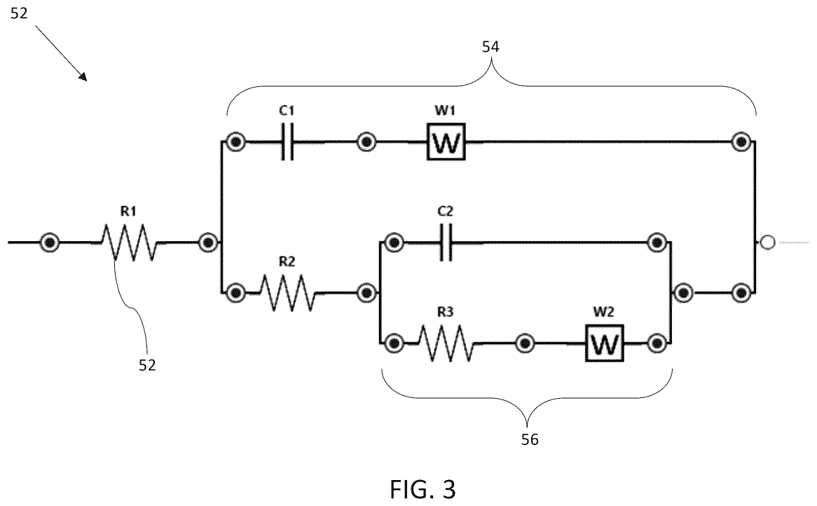

Once prepared, sensor 10 may be used to test an analyte by applying a test sample, for example a 10 μL volume, to working electrode 14 and allowed to bind with any analyte in the sample. Preferably, the test sample is localized on working electrode 14 to maximize exposure of the functionalized surface to the analyte. Once sufficient time has passed to allow the analyte to bind to working electrode 14, a conductive fluid, for example in a volume of 150 μL, is applied to sensing region 32 such that electrodes 14, 16, and 18 are in electrical communication. A voltage signal, which may have a variable voltage and/or frequency, is applied between electrodes 14 and 16, while electrode 18 is used as a reference. An output signal may be measured using a detection or reader 50 shown in FIG. 1, and then analyzed using a circuit model 52 as shown in FIG. 3.

An electrochemical circuit model 52 that may be used to analyze the electrochemical impedance of a biosensor in the presence and absence of pathogens in the sample is shown in FIG. 3. For EIS based biochemical sensors, the change in charge transfer resistance may be a useful measure as the charge transfer is typically substantially impacted by the target being bound to the sensor surface. Alternatively, the changes in capacitance C, may be measured, however the changes in capacitance are typically less dramatic.

It was found that circuit model 52 shown in FIG. 3 provides a good model to determine the sensor's response to an analyte. Circuit model 52 includes a first resistive element 52 in series with a primary RC network 54 having a primary resistor R2, a primary capacitor C1, and a nested RC network 56 in series with primary resistor R2 of primary RC network 54. Nested RC network 56 includes a secondary resistor R3 and a secondary capacitor C2. Both primary RC network 54 and nested RC network 56 include a frequency-dependent impedance component W1 and W2, respectively. Impedance component W1 in primary RC network 54 is in the capacitive branch, while impedance component W2 in nested RC network 56 is in the resistive branch. Frequency-dependent impedance components W1 and W2 may be used to compensate for the frequency response of other components in the respective RC networks 54 and 56 at different frequencies to improve the quality of the signals, and in some examples, may be designed to contribute more at low frequencies.

In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context requires that there be one and only one of the elements.

The scope of the following claims should not be limited by the preferred embodiments set forth in the examples above and in the drawings but should be given the broadest interpretation consistent with the description as a whole.

1. A sensor for detecting an analyte of interest in a fluid sample, comprising:
a substrate that supports a device architecture, the device architecture comprising a working electrode, a reference electrode and a counter electrode, the working electrode comprising a functionalized carbon surface that is functionalized to bond to the analyte of interest wherein, in response to a fluid sample applied to the working electrode that includes the analyte of interest, the device architecture generates an electrical characteristic indicative of a presence of the analyte of interest;
wherein the substrate comprises a material that is resistant to a plurality of heating cycles, each heating cycle comprising heating the substrate to a temperature of between 100 and 150° C. for at least 10 minutes.

2. The sensor of claim 1, further comprising a sensing region that is adapted to receive the fluid sample in electrical communication with the reference and counter electrode.

3. The sensor of claim 1, wherein the substrate is resistant to four or more heating cycles without substantial degradation.

4. The sensor of claim 1, wherein the functionalized carbon surface comprises amorphous carbon, carbon black, graphite, exfoliated graphite, graphene nanoplatelets, binders, stabilizers, or combinations thereof.

5. The sensor of claim 4, wherein the functionalized carbon surface is functionalized with linkers, each linker having a first terminus that is bound to the working electrode and a second terminus that is bound to a biorecognition element, each biorecognition element being selected to bind a predetermined analyte.

6. The sensor of claim 5, wherein the functionalized carbon surface is covalently functionalized with a benzoic acid-based linker.

7. The sensor of claim 6, wherein the benzoic acid-based linker comprises an ester group and are electrografted to the functionalized carbon surface.

8. The sensor of claim 7, wherein the benzoic acid-based linker is bonded to an antibody or biorecognition element.

9. The sensor of claim 8, further comprising a blocking agent that blocks a portion of the functionalized carbon surface.

10. The sensor of claim 1, further comprising a detector that is adapted to identify the electrical characteristic using electrochemical impedance spectroscopy measurement.

11. The sensor of claim 10, wherein the detector is adapted to identify the electrical characteristic by fitting a detected signal to a circuit model that comprises a primary RC network in series with a resistive element, the primary RC network comprising a primary resistor path and a primary capacitive branch, the primary resistor path comprising a primary resistor in series with a nested RC network, and the nested RC network comprising a secondary resistor in parallel with a secondary capacitor, wherein the electrical characteristic comprises a capacitance of the secondary capacitor.

12. The sensor of claim 11, wherein the detector further comprises a primary frequency-dependent impedance element in series with the primary capacitor and a secondary frequency-dependent impedance element in series with the secondary resistor.

13. A method of detecting an analyte of interest, comprising the steps of:
providing a sensor as defined in claim 1;
applying a sample solution to the working electrode and allowing the sample to interact with the functionalized surface;
applying a conductive test solution to a sensing region that includes the working electrode, the counter electrode, and reference electrodes; and
applying a test voltage between the working electrode and the counter electrode, and measuring an electrical characteristic that is indicative of the presence or absence of the analyte of interest.

14. The method of claim 13, wherein measuring the electrical characteristic comprises using a detector that comprises a primary RC network in series with a resistive element, the primary RC network comprising a primary resistor path and a primary capacitive branch, the primary resistor path comprising a primary resistor in series with a nested RC network, and the nested RC network comprising a secondary resistor in parallel with a secondary capacitor, wherein the electrical characteristic comprises a capacitance of the secondary capacitor.

15. The method of claim 14, wherein the detector further comprises a primary frequency-dependent impedance element in series with the primary capacitor and a secondary frequency-dependent impedance element in series with the secondary resistor.

16. A method of manufacturing a biosensor, comprising:
printing electrodes on a substrate using metallic ink, the electrodes comprising a working electrode, a counter electrode, and a reference electrode;
curing the electrodes on the substrate at a temperature of between 100-150° C. for at least 10 minutes;
printing a carbon surface on the working electrode using carbon-containing ink, the carbon surface being in electrical communication with the working electrode;
curing the carbon surface at a temperature of between 100-150° C. for at least 10 minutes; and
functionalizing the carbon surface to target an analyte of interest, such that the electrodes generate an electrical characteristic indicative of a presence of the analyte of interest being bonded to the functionalized carbon surface.

17. The method of claim 16, further comprising the step of applying a dielectric layer to the electrodes and curing the dielectric layer at a temperature of between 100-150° C. for at least 10 minutes.

18. The method of claim 16, wherein the functionalized carbon surface comprises a sensing region that is adapted to receive a fluid sample to be tested for the analyte of interest.

19. The method of claim 16, wherein the carbon ink comprises amorphous carbon, carbon black, graphite, exfoliated graphite, graphene nanoplatelets, binders, stabilizers, or combinations thereof.

20. The method of claim 19, wherein the functionalized carbon surface is functionalized with linkers, each linker having a first terminus that is bound to a working electrode and a second terminus that is bound to a biorecognition element, each biorecognition element being selected to bind a predetermined analyte.

21. The method of claim 20, wherein the functionalized carbon surface is covalently functionalized with a benzoic acid-based linker through diazonium reduction reaction of the linker via the substrate under applied voltage.

22. The method of claim 21, wherein the benzoic acid-based linker is chemically modified to introduce an ester group following electrografting to the functionalized carbon surface.

23. The method of claim 22, wherein the chemically modified benzoic acid-based linker is bonded to an antibody or biorecognition element, and the biosensor is incubated for at least 24 hours at a temperature of 3-8° C. or less.

24. The method of claim 23, further comprising the step of applying a blocking agent to block non-functionalized areas to reduce non-specific binding of the analyte of interest.

25. The method of claim 24, wherein applying the blocking agent comprises a 1 hr incubation time at room temperature with polyethylene glycol 8000 Da as the blocking agent.

26. The method of claim 16, wherein a detector is used to detect an electrical characteristic that is indicative of the presence or absence of the analyte of interest using electrochemical impedance spectroscopy.

27. The method of claim 16, wherein the biosensor is packaged in a sensor vessel, vacuum sealed and stored at 3-8° C. for at least 2 months.
